(19) United States
(12) Patent Application Publication
(10) Pub. No.: US 2020/0281452 A1
(43) Pub. Date: Sep. 10, 2020

(54) MEDICAL LIGHT SOURCE DEVICE

(71) Applicant: Sony Olympus Medical Solutions Inc., Tokyo (JP)

(72) Inventor: Sojung MOON, Tokyo (JP)

(73) Assignee: Sony Olympus Medical Solutions Inc., Tokyo (JP)

(21) Appl. No.: 16/722,662

(22) Filed: Dec. 20, 2019

(30) Foreign Application Priority Data
Mar. 7, 2019 (JP) 2019-041884

(51) Int. Cl. A61B 1/06; A61B 1/07; A61B 1/00

(52) U.S. Cl. CPC A61B 1/0646; A61B 1/0638; A61B 1/042; A61B 1/00006; A61B 1/07

(57) ABSTRACT

A medical light source device includes: a first light emitting device configured to emit light; an optical member provided on an optical path of the light emitted from the first light emitting device, and configured to emit a part of the light emitted by the first light emitting device in a first direction and emit rest of the light in a second direction different from the first direction; an illumination optical system provided on the optical path of the light emitted in the first direction by the optical member and configured to guide incident light to be emitted to an outside; a detector disposed on the optical path of the light emitted in the second direction by the optical member and configured to detect an amount of incident light; and an optical path changer configured to change the optical path of the light emitted from the first light emitting device in accordance with the amount of light detected by the detector.

This application claims priority from Japanese Application No. 2019-041884, filed on Mar. 7, 2019, the contents of which are incorporated by reference herein in its entirety.

### BACKGROUND

The present disclosure relates to a medical light source device.

There are known, in medical fields and industrial fields, medical devices such as an endoscope device that captures a subject image using an image sensor, and a medical microscope device (refer to JP 2016-202441 A, for example). An endoscope device, among these, includes an endoscope, an imaging device, a display device, a control device (image processing device), and a light source device, for example. In an endoscope device, illumination light is supplied from a light source device via a light guide connected to the endoscope, and the illumination light is applied to capture a subject image.

The light source device includes a light source, an optical system that guides light from the light source, and a light source including a light emitter and an optical fiber that guides light from the light emitter to the optical system. In order to protect an emitting end surface, a distal end surface of an optical fiber on the light emission side is polished to have a protrusion, an inclined surface, or a diagonal spherical shape, thereby suppressing the incident of return light of the light emitted from the own fiber. However, since the distal end surface of the optical fiber is inclined with respect to the longitudinal direction of the fiber, vignetting might occur and the desired amount of light to emit might not be ensured in some cases. To deal with this problem, JP 2016-202441 A describes a technique of providing a detecting portion for detecting the amount of light on an optical path so as to control the amount of light emitted from the light source device.

### SUMMARY

However, the detecting portion described in JP 2016-202441 A is provided outside the light source device and performs the detection processing by arranging the detection unit in the optical path, disabling detection of the amount of light during observation with the endoscope. On the other hand, providing a detecting portion using another member as disclosed in JP 2016-202441 A would complicate the configuration of the entire system.

There is a need for a medical light source device capable of detecting the amount of light emitted from the light source device even during the use of the medical device.

According to one aspect of the present disclosure, there is provided a medical light source device including: a first light emitting device configured to emit light; an optical member provided on an optical path of the light emitted from the first light emitting device, and configured to emit a part of the light emitted by the first light emitting device in a first direction and emit rest of the light in a second direction different from the first direction; an illumination optical system provided on the optical path of the light emitted in the first direction by the optical member and configured to guide incident light to be emitted to an outside; a detector disposed on the optical path of the light emitted in the second direction by the optical member and configured to detect an amount of incident light; and an optical path changer configured to change the optical path of the light emitted from the first light emitting device in accordance with the amount of light detected by the detector.

### BRIEF DESCRIPTION OF THE DRAWINGS

FIG. 1 is a schematic view illustrating a configuration of an endoscope device according to a first embodiment;

FIG. 2 is a block diagram illustrating a configuration of a camera head and a control device illustrated in FIG. 1;

FIG. 3 is a view illustrating a configuration of a light source device illustrated in FIG. 1;

FIG. 4 is a view illustrating a configuration of a light source device, which is in a state before optical axis adjustment;

FIG. 5 is a diagram illustrating illumination light control performed by the endoscope device according to the first embodiment;

FIG. 6 is a view illustrating a configuration of a light source device included in an endoscope device according to a second embodiment;

FIG. 7 is a view illustrating a configuration of a light source device included in an endoscope device according to a third embodiment;

FIG. 8 is a view illustrating a configuration of a light source device included in an endoscope device according to a fourth embodiment;

FIG. 9 is a diagram illustrating a wavelength of light emitted from the light source device according to the fourth embodiment; and

FIG. 10 is a view illustrating a configuration of a light source device included in an endoscope device according to a fifth embodiment.

### DETAILED DESCRIPTION

Hereinafter, a mode for carrying out the present disclosure (hereinafter referred to as “embodiment”) will be described. In the embodiment, a medical endoscope device that captures and displays an image inside a subject such as a patient will be described as an example of a system including a medical light source device according to the present disclosure. The present disclosure is not limited by the present embodiment. In the description of the drawings, the identical reference numerals will be used to denote identical portions.

### First Embodiment

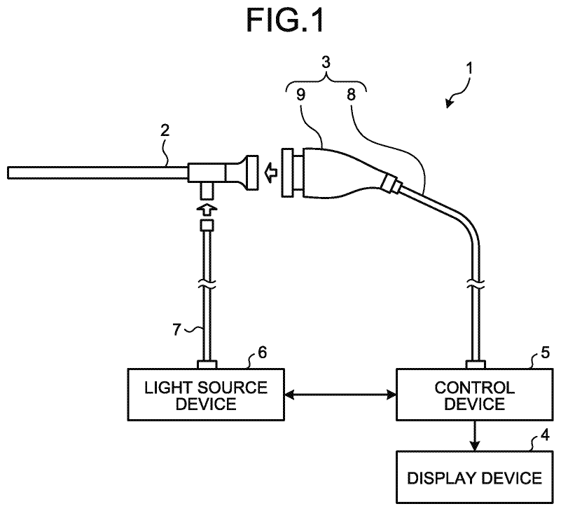

FIG. 1 is a schematic view illustrating a configuration of an endoscope device 1 according to a first embodiment. The endoscope device 1 is a device that is used in medical fields and observes a subject inside an observation target such as a person (inside a living body). As illustrated in FIG. 1, the endoscope device 1 includes an endoscope 2, an imaging device 3, a display device 4, a control device 5 (image processing device), and a light source device 6. The imaging device 3 and the control device 5 constitute a medical observation system. In the first embodiment, the endoscope 2 and the imaging device 3 constitute an image acquisition device using an endoscope such as a rigid endoscope.

The light source device 6 is connected to one end of a light guide 7, and includes: a light source unit 61 that supplies the one end of the light guide 7 with white light for illuminating the inside of the living body; and a light source controller 62 that controls emission of illumination light by the light source unit 61. The configuration of the light source device 6 will be described below. As illustrated in FIG. 1, the light source device 6 and the control device 5 may have a separate configuration and communicate with each other, or may have an integrated configuration. The light source controller 62 corresponds to an optical path changer.

The light guide 7 has one end detachably connected to the light source device 6 and the other end detachably connected to the endoscope 2. The light guide 7 transmits the light supplied from the light source device 6 from one end to the other end of the light guide 7 and supplies the light to the endoscope 2.

The imaging device 3 captures a subject image from the endoscope 2 and outputs a result of imaging. As illustrated in FIG. 1, the imaging device 3 includes a transmission cable 8 that is a signal transmission unit, and a camera head 9. In the first embodiment, the transmission cable 8 and the camera head 9 constitute a medical imaging device.

The endoscope 2 is rigid and has an elongated shape, and is inserted into a living body. The endoscope 2 internally includes an observation optical system that uses one or more lenses to focus a subject image. The endoscope 2 emits light supplied via the light guide 7 from its distal end and applies the light to the inside of the living body. The light (subject image) directed to the inside of the living body is collected by the observation optical system (lens unit 91) in the endoscope 2.

The camera head 9 is detachably connected to a proximal end of the endoscope 2. Under the control of the control device 5, the camera head 9 captures a subject image focused by the endoscope 2, and outputs an imaging signal obtained by the imaging. A detailed configuration of the camera head 9 will be described below. The endoscope 2 and the camera head 9 may have a detachable configuration as illustrated in FIG. 1, or may have an integrated configuration.

The transmission cable 8 has one end detachably connected to the control device 5 via a connector, with the other end detachably connected to the camera head 9 via a connector. Specifically, the transmission cable 8 is a cable having a plurality of electrical wires (not illustrated) disposed inside an outer jacket being an outermost layer. The plurality of electrical wires is used to transmit an imaging signal output from the camera head 9 to the control device 5, and transmit each of a control signal, a synchronization signal, a clock, and power output from the control device 5 to the camera head 9.

The display device 4 displays an image generated by the control device 5 under the control of the control device 5. The display device 4 preferably has a display unit having a size of 55 inches or more in order to easily obtain an immersive feeling during observation, but is not limited to this size.

The control device 5 processes the imaging signal input from the camera head 9 via the transmission cable 8, outputs the image signal to the display device 4, and comprehensively controls the operation of the camera head 9 and the display device 4. The detailed configuration of the control device 5 will be described below.

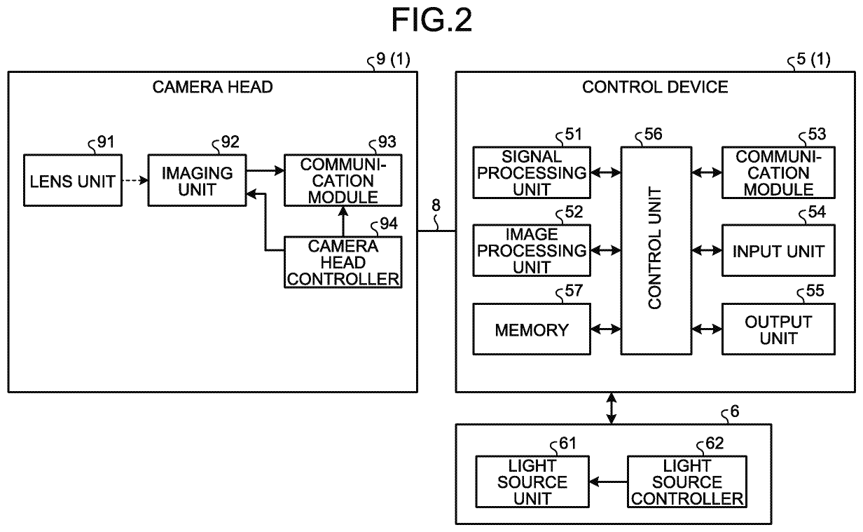

Next, configurations of the imaging device 3 and the control device 5 will be described. FIG. 2 is a block diagram illustrating a configuration of the camera head 9 and the control device 5. Note that FIG. 2 omits illustration of a connector that detachably connects the camera head 9 and the transmission cable 8.

Hereinafter, the configuration of the control device 5 and the configuration of the camera head 9 will be described in this order. The following will mainly described major portions as a configuration of the control device 5. As illustrated in FIG. 2, the control device 5 includes a signal processing unit 51, an image processing unit 52, a communication module 53, an input unit 54, an output unit 55, a control unit 56, and memory 57. The control device 5 may include a power supply unit (not illustrated) that generates a power supply voltage for driving the control device 5 and the camera head 9, supplies the generated voltage to individual portions of the control device 5 while supplying the generated voltage to the camera head 9 via the transmission cable 8.

The signal processing unit 51 performs signal processing such as noise removal and A/D conversion as necessary on an imaging signal output from the camera head 9, and thereby outputs digitized imaging signals (pulse signals) to the image processing unit 52.

The signal processing unit 51 also generates a synchronization signal and clocks for the imaging device 3 and the control device 5. A synchronization signal (for example, a synchronization signal for instructing an imaging timing of the camera head 9) or a clock (for example, a clock for serial communication) to the imaging device 3 is transmitted to the imaging device 3 by a line (not illustrated). The imaging device 3 is driven on the basis of the synchronization signal and the clock.

The image processing unit 52 generates a display image signal to be displayed on the display device 4 on the basis of the imaging signal input from the signal processing unit 51. The image processing unit 52 executes predetermined signal processing on the imaging signal, and thereby generates a display image signal including a subject image. Here, the image processing unit 52 performs known image processing including various types of image processing such as demodulation processing, interpolation processing, color correction processing, color enhancement processing, or edge enhancement processing. The image processing unit 52 outputs the generated image signal to the display device 4.

The communication module 53 outputs a signal from the control device 5 including a control signal to be described below transmitted from the control unit 56 to the imaging device 3. The communication module 53 also outputs a signal from the imaging device 3 to individual portions in the control device 5. That is, the communication module 53 is implemented as a relay device that collectively outputs the signals from the individual portions of the control device 5 to be output to the imaging device 3 by using parallel-to-serial conversion, for example, and outputs the signals input from the imaging device 3 to be distributed to individual portion of the control device 5 by using serial-to-parallel conversion, for example.

The input unit 54 is implemented as a user interface such as a keyboard, a mouse, and a touch panel, and receives inputs of various types of information.

The output unit 55 is implemented by using a speaker, a printer, a display, or the like, and outputs various types of information. The output unit 55 outputs alarm sound and alarm light, or displays an image under the control of the control unit 56.

The control unit 56 performs drive control of individual components including the control device 5 and the camera head 9, input/output control of information for individual components, or the like. The control unit 56 generates a control signal with reference to communication information data (for example, communication format information) recorded in the memory 57, and then transmits the generated control signal to the imaging device 3 via the communication module 53. The control unit 56 also outputs a control signal to the camera head 9 via the transmission cable 8.

The memory 57 is implemented by semiconductor memory such as flash memory or Dynamic Random Access Memory (DRAM), and records communication information data (for example, communication format information). The memory 57 may record various programs executed by the control unit 56.

The signal processing unit 51 may include an AF processing unit that outputs a predetermined AF evaluation value for each of frames on the basis of the input imaging signal of the frame, and an AF arithmetic unit that performs AF arithmetic processing of selecting a frame or a focus lens position most suitable as a focus position, from the AF evaluation values for each of frames output from the AF processing unit.

The above-described signal processing unit 51, the image processing unit 52, the communication module 53, and the control unit 56 are implemented by using a general-purpose processor such as a central processing unit (CPU) having internal memory (not illustrated) in which a program is recorded, or a dedicated processor such as various arithmetic circuits that executes specific function, such as an Application Specific Integrated Circuit (ASIC). The above-described units may be implemented by using a Field Programmable Gate Array (FPGA, not illustrated), a type of programmable integrated circuit. In a case where an FPGA is used, it is allowable to provide memory for storing configuration data and to achieve configuration of the FPGA which is a programmable integrated circuit on the basis of configuration data read from the memory.

Next, major portions will be mainly described, as a configuration of the camera head 9. As illustrated in FIG. 2, the camera head 9 includes a lens unit 91, an imaging unit 92, a communication module 93, and a camera head controller 94.

The lens unit 91 includes one or more lenses, and forms a subject image that has passed through the lens unit 91 on an imaging surface of an image sensor included in the imaging unit 92. The one or more lenses are movable along the optical axis. The lens unit 91 includes an optical zoom mechanism (not illustrated) that changes the angle of view by moving the one or more lenses, and a focus mechanism that changes the focal position. The lens unit 91 also forms an observation optical system that guides the observation light incident on the endoscope 2 to the imaging unit 92, together with the optical system provided in the endoscope 2.

The imaging unit 92 captures a subject image under the control of the camera head controller 94. The imaging unit 92 includes an image sensor that receives a subject image formed by the lens unit 91 and converts the image into an electrical signal. The image sensor is implemented by a charge coupled device (CCD) image sensor or a complementary metal oxide semiconductor (CMOS) image sensor. In a case where the image sensor is a CCD, for example, a signal processing unit (not illustrated) that performs signal processing (A/D conversion, etc.) on an electrical signal (analog signal) from the image sensor and outputs an imaging signal is mounted on a sensor chip or the like. In a case where the image sensor is a CMOS sensor, for example, a signal processing unit (not illustrated) that performs signal processing (A/D conversion, etc.) on an electrical signal (analog signal) obtained by converting light to the electrical signal and that outputs an imaging signal is included in the image sensor. The imaging unit 92 outputs the generated electrical signal to the communication module 93.

The communication module 93 outputs a signal transmitted from the control device 5 to individual portions in the camera head 9 such as the camera head controller 94. The communication module 93 also converts information regarding the current state of the camera head 9, or the like, into a signal format corresponding to a predetermined transmission scheme, and outputs the converted signal to the control device 5 via the transmission cable 8. That is, the communication module 93 is a relay device that distributes the signals input from the control device 5 and the transmission cable 8 by serial-to-parallel conversion, for example, and outputs the signal to individual portions of the camera head 9 as well as collectively outputting, by parallel-to-serial conversion for example, the signals from individual portion of the camera head 9 to be output to the control device 5 and the transmission cable 8.

The camera head controller 94 the controls overall operation of the camera head 9 in accordance with signals such as the drive signal input via the transmission cable 8, or an instruction signal output from an operating unit by user's operation to the operating unit such as a switch provided to be exposed on an outer surface of the camera head 9. The camera head controller 94 also outputs information regarding the current state of the camera head 9 to the control device 5 via the transmission cable 8.

The above-described communication module 93 and the camera head controller 94 are implemented by a general-purpose processor such as a CPU having internal memory (not illustrated) in which a program is recorded, and a dedicated processor such as various arithmetic circuits that execute specific functions, such as an ASIC. The above-described units may be implemented by using an FPGA, which is a type of programmable integrated circuit. Here, in a case where an FPGA is used, it is allowable to provide memory for storing configuration data and to achieve configuration of the FPGA which is a programmable integrated circuit on the basis of configuration data read from the memory.

In addition, the camera head 9 and the transmission cable 8 may include a signal processing unit that performs signal processing on an imaging signal generated by the communication module 93 or the imaging unit 92. Furthermore, an imaging clock for driving the imaging unit 92 and a control clock for the camera head controller 94 may be generated on the basis of a reference clock generated by an oscillator (not illustrated) provided in the camera head 9 so as to be each output to each of the imaging unit 92 and the camera head controller 94. Timing signals for various types of processing in the imaging unit 92 and the camera head controller 94 may be generated on the basis of the synchronization signal input from the control device 5 via the transmission cable 8 so as to be each output to each of the imaging unit 92 and the camera head controller 94. The camera head controller 94 may be provided in the transmission cable 8 or the control device 5 instead of in the camera head 9.

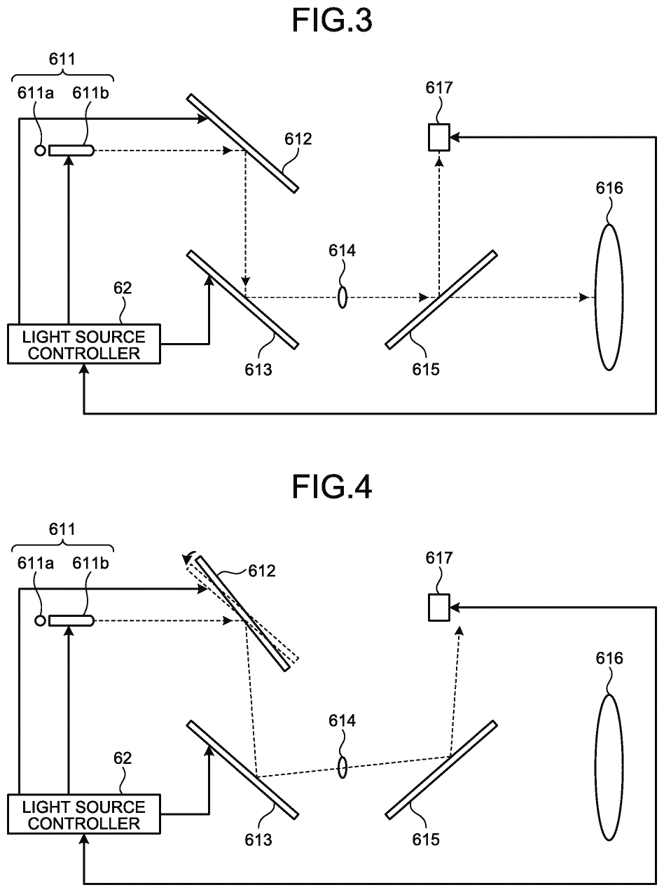

Next, the configuration of the light source device 6 will be described with reference to FIG. 3. FIG. 3 is a view illustrating a configuration of the light source device 6 illustrated in FIG. 1. The light source unit 61 includes an emission unit 611, a first mirror 612, a second mirror 613, a pinhole 614, a reflection filter 615, a condenser lens 616, and a detection unit 617. Each of these units is provided inside a casing constituting the light source device 6. In the first embodiment, the emission unit 611 corresponds to a first emission unit, the first mirror 612 and the second mirror 613 correspond to a folded optical system, the condenser lens 616 corresponds to an illumination optical system, and the reflection filter 615 corresponds to an optical member.

The emission unit 611 includes a laser light source 611a and an optical fiber 611b. White light (laser light) emitted from the laser light source 611a is guided to one end of the optical fiber 611b and emitted from the other end of the optical fiber 611b.

The first mirror 612 and the second mirror 613 reflect incident light. The first mirror 612 reflects the light emitted from the emission unit 611 (optical fiber 611b). The light reflected by the first mirror 612 is incident on the second mirror 613, which then reflects the incident light. The first mirror 612 and the second mirror 613 can alter the direction of the reflecting surface under the control of the light source controller 62. For example, the angles of the first mirror 612 and the second mirror 613 are adjusted with respect to axes extending in different directions, for example, directions orthogonal to each other. Adjustment of the angles of the first mirror 612 and the second mirror 613 uses a piezo motor, for example.

The light reflected by the second mirror 613 passes through the pinhole 614. The pinhole 614 has a hole having a preliminarily set diameter.

The reflection filter 615 folds a portion of the light traveling in an optical path in a direction different from an extending direction of the optical path and allows the rest of the light to pass. In the first embodiment, the reflection filter 615 reflects a preliminarily set proportion of light and allows the rest of the light to pass. In the first embodiment, the reflection filter 615 reflects 2% of light and allows 98% of the light to pass. The reflection filter 615 is provided in the optical path downstream of the pinhole 614 and upstream of the condenser lens 616.

The condenser lens 616 includes one or more lenses, collects the light that has passed through the reflection filter 615, and supplies the light guide 7 with the light.

The detection unit 617 includes an optical sensor, receives the light reflected by the reflection filter 615, and converts the received light into an electrical signal. The electrical signal generated by the detection unit 617 is output to the light source controller 62.

In the light source unit 61 described above, the light emitted from the emission unit 611 is reflected by the first mirror 612 and the second mirror 613, and passes through the pinhole 614. The light that has passed through the pinhole 614 is incident on the reflection filter 615. 98% of the incident light passes and 2% of the light is reflected by the detection unit 617. The light that has passed through the reflection filter 615 is collected by the condenser lens 616 so as to be incident on the light guide 7. Meanwhile, the light incident on the detection unit 617 is converted into an electrical signal (signal value) indicating a value corresponding to the amount of light.

After receiving the electrical signal from the detection unit 617, the light source controller 62 determines the necessity of adjustment of the first mirror 612 and the second mirror 613 on the basis of the signal value and a preliminarily set threshold. For example, the light source controller 62 calculates a difference between the signal value and the threshold, and determines the necessity of adjustment of the first mirror 612 and the second mirror 613 in accordance with the difference. The threshold is a value set, for example, as a value corresponding to a maximum received amount of light predicted from the output of the emission unit 611 (laser light source 611a), the amount of light passing through the pinhole 614, and the reflection characteristics of the reflection filter 615. The maximum received amount of light is the amount of light detected when the optical axis of light traveling in the optical path passes through the best position. An example of the best position would be a position where the optical axis passes through the center of the pinhole 614 (hole). This maximum received amount of light corresponds to a target amount of light.

In a case where the light source controller 62 determines that adjustment of the first mirror 612 and the second mirror 613 is necessary, the light source controller 62 changes the angle of the first mirror 612 and/or the second mirror 613 in accordance with the magnitude of the difference. The movements of the first mirror 612 and the second mirror 613 are preliminarily associated with each other. In a case where the target amount of light has not reached by performing angle alteration processing one time, the light source controller 62 adjusts the angle to obtain the maximum received amount of light by repeatedly performing the angle alteration processing.

FIG. 4 is a view illustrating a configuration of a light source device, which is in a state before optical axis adjustment. As illustrated in FIG. 4, in a case where the optical path deviates and the amount of light to be incident on the detection unit 617 or the condenser lens 616 is small or even no light is incident on the detection unit 617 or the condenser lens 616, the amount of light to be supplied from the light source device 6 to the light guide 7 would be below a set amount. In this case, the light source controller 62 changes the angle of the first mirror 612 and/or the second mirror 613 on the basis of the electrical signal from the detection unit 617. In the example illustrated in FIG. 4, the angle is changed in the first mirror 612 alone (refer to the broken line superimposed on the first mirror 612). Changing the angle of the first mirror 612 under the control of the light source controller 62 will adjust the optical axis of the light emitted from the emission unit 611 (refer to FIG. 3).

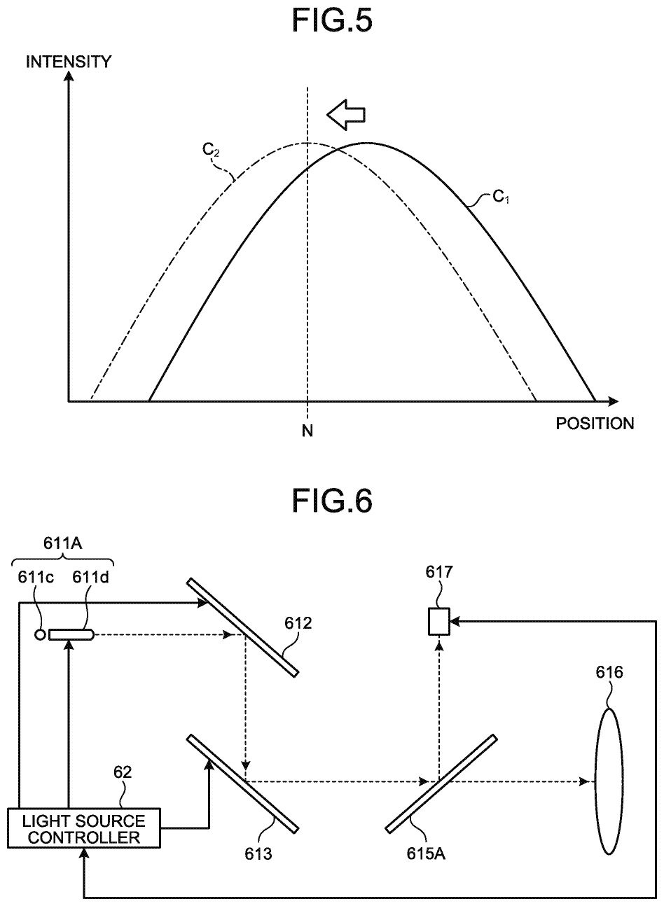

FIG. 5 is a diagram illustrating illumination light control performed by the endoscope device according to the first embodiment. In a case where the optical axis deviates from the center of the optical path (axis N), where the optical axis should pass, such as when the optical axis of the emitted light is inclined with respect to the longitudinal direction of the optical fiber, due to the shape of the light emitting end of the emission unit 611 (optical fiber 611b), the peak of the light distribution (distribution C₁ illustrated in FIG. 5) would deviate from the axis N. Traveling through the optical path with deviation in the light distribution (optical axis) would cause problems such as unnecessary level of vignetting of the light at the pinhole 614 or occurrence of non-collection of the light by the condenser lens 616, leading to a failure in ensuring the intensity as the illumination light. In the first embodiment, the light source controller 62 alters the angles of the first mirror 612 and the second mirror 613 on the basis of the detection result of the detection unit 617. This will shift the peak of the light distribution to the axis N side (distribution C₂ illustrated in FIG. 5). In this manner, the optical axis of the light is controlled so as to approach the ideal optical path to pass through, it is possible to make adjustment so as to ensure the intensity as the illumination light.

In the first embodiment described above, the reflection filter 615 is used to allow a part of the light guided to the condenser lens 616 to be incident on the detection unit 617. This makes it possible to detect the amount of light emitted from the light source device 6 even during the use of the endoscope.

According to the first embodiment described above, the angles of the first mirror 612 and the second mirror 613 are controlled on the basis of the detection result of the detection unit 617. This controls the optical axis of the light so as to approach the ideal optical path to pass through, making it possible to achieve adjustment so as to ensure the intensity as illumination light.

### Second Embodiment

Next, a second embodiment will be described. FIG. 6 is a view illustrating a configuration of a light source device included in an endoscope device according to the second embodiment. In the second embodiment, the configuration other than the light source unit 61 is the same as that of the endoscope device 1 of the first embodiment described above, and thus the description thereof will be omitted.

The light source unit illustrated in FIG. 6 includes an emission unit 611A, the first mirror 612, the second mirror 613, a reflection filter 615A, the condenser lens 616, and a detection unit 617. Differences in the light source unit according to the second embodiment from the light source unit 61 according to the first embodiment exist in the light emitted from the emission unit 611A and the reflection characteristics of the reflection filter 615A. Other configurations are the same as those in the first embodiment. Hereinafter, portions different from the first embodiment will be described. In the second embodiment, the emission unit 611A corresponds to the first emission unit, and the reflection filter 615A corresponds to an optical member.

The emission unit 611A includes a laser light source 611c and an optical fiber 611d, and light (laser light) emitted from a laser light source 611c is guided to one end of an optical fiber 611d and emitted from the other end of the optical fiber 611d. The emission unit 611A emits light (infrared light) in a wavelength band of 500 nm or more and 700 nm or less.

The reflection filter 615A reflects light of a preliminarily set wavelength or less. In the second embodiment, the reflection filter 615A reflects light having a wavelength of 600 nm or less and allows light having a wavelength greater than 600 nm to pass.

The detection unit 617 receives the light reflected by the reflection filter 615A and converts the received light into an electrical signal. The electrical signal generated by the detection unit 617 is output to the light source controller 62.

After receiving the electrical signal from the detection unit 617, the light source controller 62 determines the necessity of adjustment of the first mirror 612 and the second mirror 613 on the basis of the signal value and a preliminarily set threshold. For example, the light source controller 62 calculates a difference between the signal value and the threshold, and determines the necessity of adjustment of the first mirror 612 and the second mirror 613 in accordance with the difference. The threshold is a value set, for example, as a value corresponding to a maximum received amount of light, with the wavelength band of 500 nm or more and 600 nm or less, predicted from the output of the emission unit 611A (laser light source 611c) and from the reflection characteristics of the reflection filter 615A. Specifically, the light source controller 62 changes the angle of the first mirror 612 and/or the second mirror 613 in accordance with the magnitude of the difference. The movements of the first mirror 612 and the second mirror 613 are preliminarily associated with each other.

In the second embodiment described above, the reflection filter 615 is used to allow a part of the light guided to the condenser lens 616 to be incident on the detection unit 617. This makes it possible to detect the amount of light emitted from the light source device 6 even during the use of the endoscope.

Similarly to the first embodiment, according to the second embodiment described above, the angles of the first mirror 612 and the second mirror 613 are controlled on the basis of the detection result of the detection unit 617. This makes it possible to control the optical axis of the light so as to approach the ideal optical path to pass through, enabling adjustment so as to ensure the intensity as illumination light.

In a case where white light is emitted from the endoscope 2 in the second embodiment described above, the light source unit 61 according to the first embodiment described above may be provided separately, or white light may be directly supplied from a light source that emits white light to the light guide. The light source that emits white light is not limited to a laser light source, and may use a semiconductor light source such as a light emitting diode (LED) light source.

### Third Embodiment

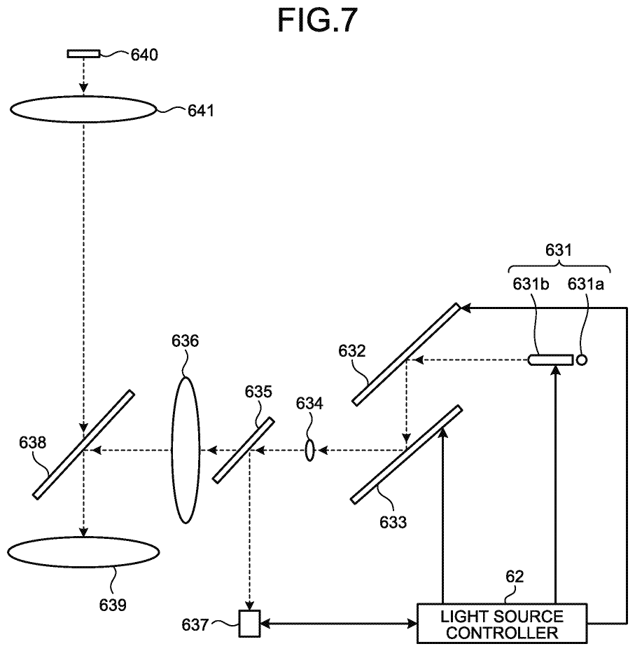

Next, a third embodiment will be described. FIG. 7 is a view illustrating a configuration of a light source device included in an endoscope device according to the third embodiment. In the third embodiment, the configuration other than the light source unit 61 is the same as that of the endoscope device 1 of the first embodiment described above, and thus the description thereof will be omitted.

The light source unit illustrated in FIG. 7 includes a first emission unit 631, a first mirror 632, a second mirror 633, a pinhole 634, a first reflection filter 635, a first collimating lens 636, and a detection unit 637, a second reflection filter 638, a condenser lens 639, a second emission unit 640, and a second collimating lens 641. In the third embodiment, the first emission unit 631 corresponds to a first emission unit, the first mirror 632 and the second mirror 633 correspond to a folded optical system, the condenser lens 639 corresponds to the illumination optical system, the first reflection filter 635 corresponds to an optical member, and the second emission unit 640 corresponds to a second emission unit. The first collimating lens 636, the second reflection filter, and the second collimating lens 641 are appropriately arranged in accordance with the optical system to be formed.

The first emission unit 631 includes a laser light source 631a and an optical fiber 631b, and light (laser light) emitted from the laser light source 631a is guided to one end of the optical fiber 631b and emitted from the other end of the optical fiber 631b. The first emission unit 631 emits light (infrared light) in a wavelength band of 500 nm or more and 700 nm or less.

The first mirror 632 and the second mirror 633 reflect incident light. The first mirror 632 reflects the light emitted from the first emission unit 631 (optical fiber 631b). The light reflected by the first mirror 632 is incident on the second mirror 633, which then reflects the incident light. The first mirror 632 and the second mirror 633 can change the direction of the reflecting surface under the control of the light source controller 62. For example, the angles of the first mirror 632 and the second mirror 633 are adjusted with respect to axes extending in different directions, for example, directions orthogonal to each other. Adjustment of the angles of the first mirror 632 and the second mirror 633 uses a piezo motor, for example.

The light reflected by the second mirror 633 passes through the pinhole 634. The pinhole 634 has an aperture having a preliminarily set diameter.

The first reflection filter 635 reflects light having a preliminarily set wavelength or less. In the third embodiment, the first reflection filter 635 reflects light having a wavelength of 600 nm or less and allows light having a wavelength greater than 600 nm to pass. The first reflection filter 635 is provided downstream of the pinhole 634 and upstream of the second reflection filter 638 in the optical path.

The first collimating lens 636 includes one or more lenses, and collimates the light that has passed through the first reflection filter 635 and guides the collimated light to the second reflection filter 638.

The detection unit 637 includes an optical sensor, receives light reflected by the first reflection filter 635, and converts the light into an electrical signal. The electrical signal generated by the detection unit 637 is output to the light source controller 62.

The second reflection filter 638 reflects light having a preliminarily set wavelength or less. In the third embodiment, the second reflection filter 638 reflects light having a wavelength greater than 600 nm and allows light having a wavelength of 600 nm or less to pass. In a case where the light emission periods of the first emission unit 631 and the second emission unit 640 overlap with each other, the second reflection filter 638 functions as a combining member that combines the light beams emitted from individual units.

The condenser lens 639 includes one or more lenses, and collects the light emitted from the first emission unit 631 and reflected by the second reflection filter 638, and the light emitted from the second emission unit 640 and that has passed through the second reflection filter 638, and then, supplies the collected light to the light guide 7.

The second emission unit 640 includes an LED light source that emits white light. The light emitted from the second emission unit 640 has a phase variation larger than the phase variation of the light emitted from the first emission unit 631.

The second collimating lens 641 includes one or more lenses, and collimates the light emitted from the second emission unit 640 and guides the collimated light to the second reflection filter 638.

In the light source unit described above, the light emitted from the first emission unit 631 is reflected by the first mirror 632 and the second mirror 633 and passes through the pinhole 634. The light that has passed through the pinhole 634 is incident on the first reflection filter 635. A part of the incident light passes through, and the rest of the light is reflected by the detection unit 637. The light that has passed through the first reflection filter 635 passes through the first collimating lens 636 and the second reflection filter 638 and collected by the condenser lens 639, so as to be incident on the light guide 7. Meanwhile, the light incident on the detection unit 637 is converted into an electrical signal (signal value) indicating a value corresponding to the amount of light.

The light emitted from the second emission unit 640 passes through the second collimating lens 641 and the second reflection filter 638, and collected by the condenser lens 639, so as to be incident on the light guide 7.

In the third embodiment, under the control of the light source controller 62, one of observation modes, that is, either an infrared observation mode of performing observation with the light from the first emission unit 631 or a normal observation mode of performing observation with the light from the second emission unit 640 is selected. That is, in the third embodiment, either light having a wavelength band greater than 600 nm out of the light emitted from the first emission unit 631 and light having a wavelength band of 600 nm or less out of the light emitted from the second emission unit 640 is to be incident on and collected by the condenser lens 639, so as to be incident on the light guide 7.

After receiving the electrical signal from the detection unit 637 in the infrared observation mode, the light source controller 62 determines the necessity of adjustment of the first mirror 632 and the second mirror 633 on the basis of the signal value and a preliminarily set threshold. For example, the light source controller 62 calculates a difference between the signal value and the threshold, and determines the necessity of adjustment of the first mirror 632 and the second mirror 633 in accordance with the difference. The threshold is a value set, for example, as a value corresponding to a maximum received amount of light predicted from the output of the first emission unit 631 (laser light source), the amount of light passing through the pinhole 634, and the reflection characteristics of the first reflection filter 635. Specifically, the light source controller 62 changes the angle of the first mirror 632 and/or the second mirror 633 in accordance with the magnitude of the difference. The movements (inclination angle) of the first mirror 632 and the second mirror 633 are preliminarily associated with each other.

In the third embodiment described above, the first reflection filter 635 is used to allow a part of the light guided from the first emission unit 631 toward the condenser lens 639 to be incident on the detection unit 637. This makes it possible to detect the amount of light emitted from the light source device 6 even during the use of the endoscope.

According to the third embodiment described above, the angles of the first mirror 632 and the second mirror 633 are controlled on the basis of the detection result of the detection unit 637. This makes it possible to control the optical axis of the light so as to approach the ideal optical path to pass through, enabling adjustment so as to ensure the intensity as illumination light.

### Fourth Embodiment

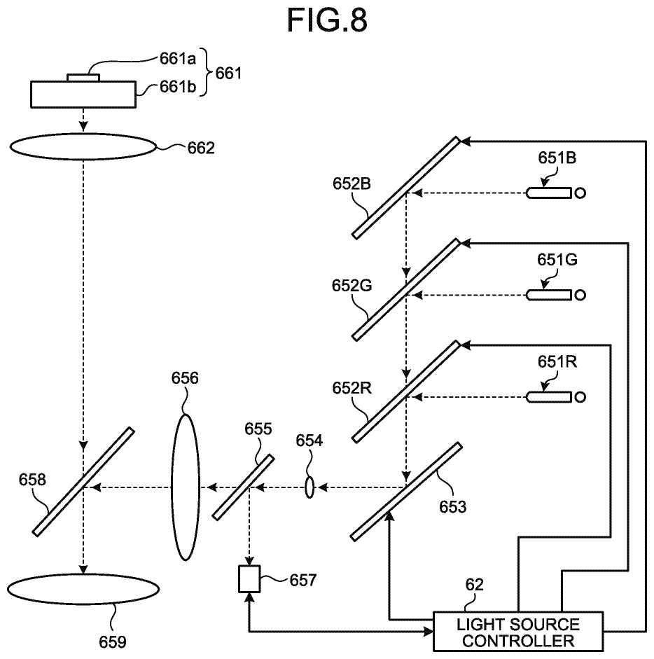

Next, a fourth embodiment will be described. FIG. 8 is a view illustrating a configuration of a light source device included in an endoscope device according to the fourth embodiment. In the fourth embodiment, the configuration other than the light source unit 61 is the same as that of the endoscope device 1 of the first embodiment described above, and thus the description thereof will be omitted.

The light source unit illustrated in FIG. 8 includes a plurality of first light sources (blue light source 651B, green light source 651G, red light source 651R), a first mirror 652B, a second mirror 652G, a third mirror 652R, a fourth mirror 653, a pinhole 654, a first reflection filter 655, a first collimating lens 656, a detection unit 657, a second reflection filter 658, a condenser lens 659, a second emission unit 661, and a second collimating lens 662. In the fourth embodiment, the first light source corresponds to the first emission unit, and the first mirror 652B, the second mirror 652G, the third mirror 652R, and the fourth mirror 653 correspond to a folded optical system, the first reflection filter 655 corresponds to an optical member, the second emission unit 661 corresponds to a second emission unit, the second reflection filter 658 corresponds to a combining member, and the condenser lens 659 corresponds to an illumination optical system. The first collimating lens 656 and the second collimating lens 662 are appropriately arranged in accordance with the optical system to be formed.

Each of the first light sources includes a laser light source and an optical fiber. Light (laser light) emitted from the laser light source is guided to one end of the optical fiber and emitted from the other end of the optical fiber.

The blue light source 651B emits light (blue light) in a wavelength band of 450 nm or more and 500 nm or less.

The green light source 651G emits light (green light) in a wavelength band of 490 nm or more and 600 nm or less.

The red light source 651R emits light (red light) in a wavelength band of 590 nm or more and 750 nm less.

First mirror 652B, second mirror 652G, third mirror 652R, and fourth mirror 653 reflect incident light. The first mirror 652B reflects light in the wavelength band of 450 nm or more and 490 nm or less. The second mirror 652G reflects light in the wavelength band of 490 nm or more and 590 nm or less and passes light in the wavelength band of 450 nm or more and 490 nm or less. The third mirror 652R reflects light in the wavelength band of 590 nm or more and 750 nm or less and allows light in the wavelength band of 450 nm or more and 590 nm or less to pass. Beams of the light that has passed through the third mirror 652R and the light that has been reflected by the third mirror 652R are incident on the fourth mirror 653, and the incident light is reflected by the fourth mirror 653 toward the pinhole 654. The first mirror 652B, the second mirror 652G, the third mirror 652R, and the fourth mirror 653 can alter the direction of the reflecting surface under the control of the light source controller 62. For example, the angles of the first mirror 652B, the second mirror 652G, the third mirror 652R, and the fourth mirror 653 are adjusted with respect to a preliminarily set axis. Adjustment of the angles of each of the mirrors uses a piezo motor, for example.

The light reflected by the fourth mirror 653 passes through the pinhole 654. The pinhole 634 has an aperture having a preliminarily set diameter.

The first reflection filter 655 reflects light having a preliminarily set wavelength or less. In the fourth embodiment, the first reflection filter 655 reflects light of a part of the wavelength band of green light and allows light of other wavelength bands to pass. The first reflection filter 655 is provided downstream of the pinhole 654 and upstream of the second reflection filter 658 in the optical path.

The first collimating lens 656 includes one or more lenses, and collimates the light that has passed through the first reflection filter 655 and guides the collimated light to the second reflection filter 658.

The detection unit 657 includes an optical sensor, receives light reflected by the first reflection filter 655, and converts the light into an electrical signal. The electrical signal generated by the detection unit 657 is output to the light source controller 62.

The second reflection filter 658 includes a half mirror, for example, and reflects half of the incident light, and allows the rest of the light to pass. The second reflection filter 658 combines the light emitted from the first light source and the light emitted from the second emission unit 661.

The condenser lens 659 includes one or more lenses, and collects light that has passed through the first collimating lens 656 and light that has been emitted from the second emission unit 661 and has passed through the second reflection filter 658, and supplies the collected light to the light guide 7.

The second emission unit 661 includes: an LED light source 661a that emits blue light; and a yellow phosphor 661b that emits yellow (570 nm or more and 590 nm or less) fluorescence. The second emission unit 661 combines the blue light and yellow light to emit white light. The light emitted from the second emission unit 661 has a phase variation larger than the phase variation of the light emitted from the first light source (blue light source 651B, green light source 651G, red light source 651R).

The second collimating lens 662 includes one or more lenses, and collimates the light emitted from the second emission unit 661 and guides the collimated light to the second reflection filter 658.

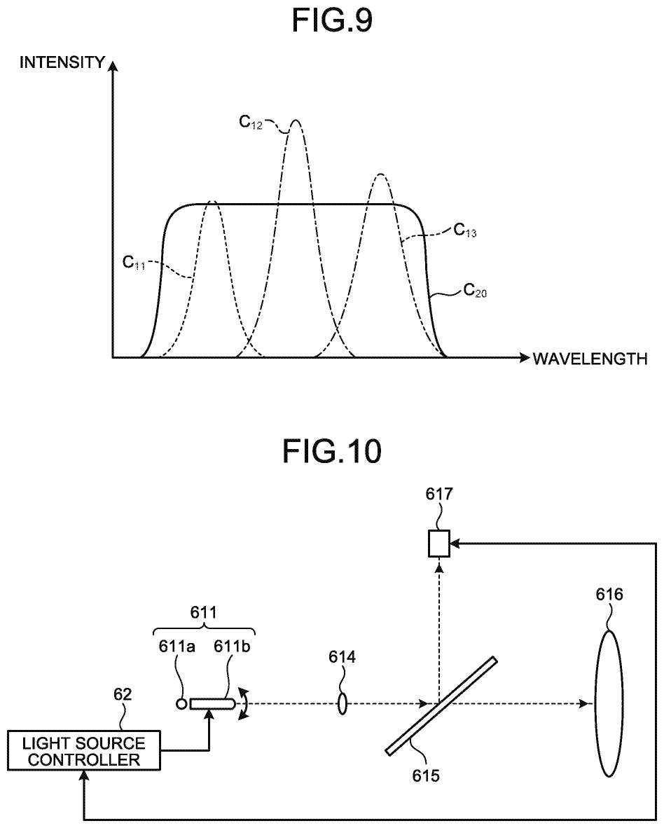

FIG. 9 is a diagram illustrating a wavelength of light emitted from the light source device according to the fourth embodiment. In FIG. 9, the horizontal axis indicates the wavelength, and the vertical axis indicates the light intensity.

The light emitted from the first light source (blue light source 651B, green light source 651G, and red light source 651R), specifically the light incident on the pinhole 654, is intermittent light depending on the wavelength band of the light emitted by each of the light sources. For example, FIG. 9 illustrates a waveform C₁₁ corresponding to the blue light emitted from the blue light source 651B, a waveform C₁₂ corresponding to the green light emitted from the green light source 651G, and a waveform C₁₃ corresponding to the red light emitted from the red light source 651R.

The light emitted from the second emission unit 661, specifically, the light incident on the second collimating lens 662, is light in a broad wavelength band. For example, FIG. 9 illustrates a waveform C₂₀ corresponding to white light emitted from the second emission unit 661.

In a case where the first light source (blue light source 651B, green light source 651G, red light source 651R) and the second emission unit 661 are driven simultaneously, the light produced by combining light beams of waveforms C₁₁, C₁₂, C₁₃, and C₂₀ as illustrated in FIG. 9 will be incident on the light guide 7. The light emitted from the second emission unit 661 interpolates intermittent light emitted from the first light source.

In the light source unit described above, the light emitted from a first emission unit 651 passes through the first mirror 652B, the second mirror 652G, and the third mirror 652R, reflected by the fourth mirror 653, and then passes through the pinhole 654. The light that has passed through the pinhole 654 is incident on the first reflection filter 655. A part of the incident light passes through, and the rest of the light is reflected by the detection unit 657. The light that has passed through the first reflection filter 655 passes through the first collimating lens 656 and the second reflection filter 658 and collected by the condenser lens 659, so as to be incident on the light guide 7. Meanwhile, the light incident on the detection unit 657 is converted into an electrical signal (signal value) indicating a value corresponding to the amount of light.

The light emitted from the second emission unit 661 passes through the second collimating lens 662 and the second reflection filter 658, and collected by the condenser lens 659, so as to be incident on the light guide 7.

After receiving the electrical signal from the detection unit 657 in the infrared observation mode, the light source controller 62 determines the necessity of adjustment of the first mirror 652B, the second mirror 652G, the third mirror 652R, and the fourth mirror 653 on the basis of the signal value and a preliminarily set threshold. For example, the light source controller 62 calculates a difference between the signal value and the threshold, and determines the necessity of adjustment of each of the mirrors in accordance with the difference. The threshold is a value set, for example, as a value corresponding to a maximum received amount of light predicted from the output of the first emission unit (laser light source), the amount of light passing through the pinhole 654, and the reflection characteristics of the first reflection filter 655. Specifically, the light source controller 62 changes the angle of each of the first mirror 652B, the second mirror 652G, the third mirror 652R, and/or the fourth mirror 653 in accordance with the magnitude of the difference.

In the fourth embodiment described above, the first reflection filter 655 is used to cause a part of light guided from the first light source (blue light source 651B, green light source 651G, red light source 651R) toward the condenser lens 659 to be incident on the detection unit 657. Therefore, the amount of light emitted from the light source device 6 can be detected even during the use of the endoscope.

According to the fourth embodiment described above, the angle of each of the first mirror 652B, the second mirror 652G, the third mirror 652R, and/or the fourth mirror 653 is controlled on the basis of the detection result of the detection unit 657. This makes it possible to control the optical axis of the light so as to approach the ideal optical path to pass through, enabling adjustment so as to ensure the intensity as illumination light.

While the above-described fourth embodiment is an example of controlling the angles of the first mirror 652B, the second mirror 652G, the third mirror 652R, and the fourth mirror 653 on the basis of the detection result of the detection unit 657, it is allowable to configure to adjust the angle of at least one mirror.

In addition, the fourth embodiment described above is an example in which light having a part of the wavelength band of green light is incident on the detection unit 657 and then the detection unit 657 outputs an electrical signal based on this light. Alternatively, however, it is also allowable to have a configuration in which the first reflection filter 655 reflects 2% of the light that has passed through the pinhole 654 and causes the rest of the light to pass, and the detection unit 657 receives the part of the light including light of all wavelengths emitted by the first light source.

In addition, it is allowable to have a configuration in which the detection unit 657 includes an image sensor or the like that can receive light two-dimensionally so as to detect a two-dimensional light distribution for the light that has passed through the pinhole 654. With a capability of the image sensor to receive light for each of colors, it is also possible to detect the light intensity or the like for each of the colors and individually control the first mirror 652B, the second mirror 652G, and the third mirror 652R.

### Fifth Embodiment

Next, a fifth embodiment will be described. FIG. 10 is a view illustrating a configuration of a light source device included in an endoscope device according to the fifth embodiment. In the fifth embodiment, the configuration other than the light source unit 61 is the same as that of the endoscope device 1 of the first embodiment described above, and thus the description thereof will be omitted.

The light source unit illustrated in FIG. 10 includes an emission unit 611, a pinhole 614, a reflection filter 615, a condenser lens 616, and a detection unit 617. The light source unit according to the fifth embodiment is different from the light source unit 61 according to the first embodiment in that the first mirror 612 and the second mirror 613 are not provided. Other configurations are the same as those in the first embodiment. Hereinafter, portions different from the first embodiment will be described.

The emission unit 611 causes light to be incident on the pinhole 614. Moreover, in the emission unit 611, the direction of the emitting end of the optical fiber 611b is altered under the control of the light source controller 62. In the emission unit 611, the angle of the longitudinal axis of the optical fiber 611b is changed or the light emitting position is changed by moving the laser light source 611a and the optical fiber 611b, under the control of the light source controller 62, for example.

After receiving the electrical signal from the detection unit 617, the light source controller 62 determines the necessity of adjustment of the emission unit 611 on the basis of the signal value and a preliminarily set threshold. For example, similarly to the first embodiment, the light source controller 62 calculates a difference between the signal value and the threshold and determines the necessity of adjustment of the emission unit 611 in accordance with the difference. When the light source controller 62 determines that adjustment of the emission unit 611 is necessary, the light source controller 62 controls the light emitting direction of the emission unit 611.

In the fifth embodiment described above, the reflection filter 615 is used to allow a part of the light guided to the condenser lens 616 to be incident on the detection unit 617, similarly to the first embodiment. This makes it possible to detect the amount of light emitted from the light source device 6 even during the use of the endoscope.

In addition, according to the fifth embodiment described above, the direction of the light emitted from the emission unit 611 is controlled on the basis of the detection result of the detection unit 617. This makes it possible to control the optical axis of the light so as to approach the ideal optical path to pass through, enabling adjustment so as to ensure the intensity as illumination light.

While the above is description of the modes for carrying out the present disclosure, the present disclosure should not be limited by only the embodiments described above. In the above-described embodiment, the control device 5 performs signal processing or the like. Alternatively, however, signal processing or the like may also be performed on the camera head 9 side.

The first to fourth embodiments described above are an example in which the reflection filter that guides light to the detection unit reflects light. Alternatively, however, it is also allowable to have a configuration in which the light in a specific direction is directed to be reflected or pass through.

While the first, third, and fourth embodiments described above are an example in which a pinhole is provided to detect the intensity of light that has passed through the pinhole, it is also allowable to omit a pinhole as described in the second embodiment. Moreover, a pinhole may be provided in the second embodiment.

The first to fourth embodiments described above are an example of adjusting angles of mirrors. However, it is also allowable to alter the light emitting position and angle of the emission unit instead of adjusting angles of mirrors.

Moreover, the first to fourth embodiments described above are an example in which the light reflected by the mirror is detected by the detection unit and the light transmitted through the mirror is incident on the condenser lens. However, it is also allowable to have a configuration in which the light reflected by the mirror is incident on the condenser lens and the light transmitted through the mirror is detected by the detection unit.

As described above, the endoscope light source device according to the present disclosure has advantages in adjusting the amount of light emitted from the light source device with a simple configuration.

According to the present disclosure, it is possible to detect the amount of light emitted from the light source device even during the use of the medical device.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A medical light source device comprising:
a first light emitting device configured to emit light;
an optical member provided on an optical path of the light emitted from the first light emitting device, and configured to emit a part of the light emitted by the first light emitting device in a first direction and emit rest of the light in a second direction different from the first direction;
an illumination optical system provided on the optical path of the light emitted in the first direction by the optical member and configured to guide incident light to be emitted to an outside;
a detector disposed on the optical path of the light emitted in the second direction by the optical member and configured to detect an amount of incident light; and
an optical path changer configured to change the optical path of the light emitted from the first light emitting device in accordance with the amount of light detected by the detector.

2. The medical light source device according to claim 1, further comprising
a folded optical system provided between the first light emitting device and the optical member and configured to fold the light emitted by the first light emitting device and guide the light to the optical member,
wherein the first light emitting device includes: a light emitter configured to emit light of a predetermined wavelength band; and a light source including an optical fiber configured to guide the light from the light emitter to the optical member, and
the optical path changer is configured to alter a light folded mode by the folded optical system.

3. The medical light source device according to claim 1,
wherein the first light emitting device includes: a light emitter configured to emit light of a predetermined wavelength band; and a light source including an optical fiber configured to guide the light from the light emitter to the optical member, and
the optical path changer is configured to alter an emission direction of the light emitted from the first light emitting device.

4. The medical light source device according to claim 1, wherein the first light emitting device includes:
a first light source including
a first light emitter configured to emit light of a predetermined wavelength band, and
a first optical fiber configured to guide the light from the first light emitter to the optical member; and
a second light source including
a second light emitter configured to emit light of a wavelength band different from the wavelength band of the light emitted from the first light source, and
a second optical fiber configured to guide the light from the second light emitter to the optical member, and
the optical member is configured to emit a part of the light emitted from the first light source and from the second light source in the second direction.

5. The medical light source device according to claim 4,
wherein the optical member is configured to emit light of a part of the wavelength band of the light emitted from the first light source and the second light source, in the second direction.

6. The medical light source device according to claim 4,
wherein the optical member is configured to emit a predetermined proportion of the light emitted from the first light source and the second light source, in the second direction.

7. The medical light source device according to claim 1, further comprising:
a second light emitting device configured to emits light having a larger phase variation than the light emitted by the first light emitting device; and
a combining member configured to combine the light emitted from the first light emitting device and the light emitted from the second light emitting device so as to be incident on the illumination optical system,
wherein the optical member is provided upstream of the combining member.

8. The medical light source device according to claim 1, further comprising
a pinhole provided upstream of the optical path of the optical path changer and formed with a preliminarily set diameter, through which the optical path passes.
